(19) United States
(12) Patent Application Publication
(10) Pub. No.: US 2006/0105032 A1
(43) Pub. Date: May 18, 2006

(54) Multiple sclerosis treatment

(76) Inventors: Marina Anetta Lynch, Dublin (IE); Yvonne Mairead Nolan, Cork (IE); Anthony Ernest Bolton, Shannon (IE); Arkady Mandel, Mississauga (CA)

Correspondence Address: Karen E. Flick;Foley & Lardner LLP, 1530 Page Mill Road, Palo Alto, CA, 94304-1125, US

(21) Appl. No.: 11/227,984

(22) Filed: Sep. 14, 2005

(30) Foreign Application Priority Data
Sep. 15, 2004 (IE) S2004/0613

(51) Int. Cl. A61K 9/127

(52) U.S. Cl. 424/450; 977/907

(57) ABSTRACT

Symptoms, including biochemical correlates, of multiple sclerosis (MS) in a mammal are beneficially affected by administering to the mammal small doses of bodies, such as liposomes, of a size resembling that of mammalian cells, the bodies having phosphate glycerol head groups presented exteriorly on their surfaces. Preferred are liposomes comprised of 50-100% phosphatidylglycerol, with the phospho glycerol headgroups thereof exteriorly presented.

### CROSS-REFERENCE TO RELATED PATENT APPLICATIONS

This application claims the benefit of priority under 35 U.S.C. §119(a) to Irish application serial number S2004/0613, filed 15 Sep. 2004.

### FIELD OF THE INVENTION

This invention relates to medicinal compositions, their preparation and their use in the prophylaxis against, and the alleviation and treatment of disorders of the central nervous system. More specifically, it relates to compositions for use in prophylaxis against and alleviation and treatment of multiple sclerosis.

### BACKGROUND OF THE INVENTION

Multiple sclerosis (MS) is a disease of the central nervous system (CNS), namely brain and spinal cord. It is predominantly a disease of temperate latitudes, and of the western hemisphere. Regions north of 40° latitude (Northern Europe, Scandinavia, British Isles, Northern USA, Canada) have a relatively high prevalence of MS, with certain localized areas within these territories having an incidence of 200-250 cases per 100,000 of population. The reasons for the uneven distribution of MS around the world is currently not well understood.

MS tends to manifest itself primarily in human patients 30-45 years of age, although it is certainly not confined to this age group. It tends to be more prevalent in females than in males. Its causes are currently unknown. There appears to be a genetic factor at work, in the sense that a family history of MS indicates a higher risk of contracting the disorder. The uneven distribution of MS prevalence geographically suggests that there may be environmental contributing factors and/or dietary contributing factors. There is strong evidence that MS is an autoimmune disease, i.e. a disorder of the immune system where cells which are responsible for identifying and destroying harmful pathogens invading the body mistakenly identify and attack a component of the body's own tissues. This proposition is not universally accepted, however, and viral and bacterial causes (e.g. human herpes virus 6, Epstein-Barr virus and Chlamydia Pneumonia bacterium) have been proposed as alternative explanations. It is not a contagious disease.

MS is a disease predominantly of the white matter of the CNS. The white matter is made up of neurons, the function of which is to transmit communication signals internally within the CNS and between the CNS and the nerves supplying the rest of the body. These white matter neurons are long thin cells having a bulbous head (soma) containing the cell nucleus, and an elongated strand, the axon, which is coated with a myelin sheath. From the soma extend a large number of branched tendrils, known as dendrites. The axon of one neuron is connected to the dendrites of other neurons through connections known as synapses, so that nerve impulses can travel along the axon and thence to other neurons via chemical signals (neurotransmitters) moving across the synapse. A damaged myelin sheath, an improperly operating synapse and a lack of neurotransmitters can all impede the required transmission of nerve impulses to the appropriate parts of the body.

Oligodendrocytes, a type of glial (maintenance) cell, are associated with axons. The function of the oligodendrocytes is understood to be creation and repair of the myelin sheath of the axons, and the feeding of essential factors to the axons. Each oligodendrocyte is associated with several axons, and each axon in the properly functioning system is maintained by several oligodendrocytes. It is becoming increasingly well accepted that loss or dysfunction of oligodendrocytes is a significant factor in development and progression of MS.

There may also be a role played by microglia in MS. Microglia are a type of glial cell whose function is to remove dead cells and other debris from the CNS. Microglial cells are the brain-resident macrophages and function like macrophages in the periphery, for example, among other functions, they present antigen and they can release pro-inflammatory factors including cytokines. The microglia can act as antigen-presenting cells in MS, thus exacerbating an autoimmune response. Some researchers have noted that microglial cells play an important role in initiating and maintaining CNS autoimmune injury. In addition, activated microglia increase recruitment of immune cells to the site of injury, as well as release of cytotoxic or inflammatory mediators. In experimental autoimmune encephalomyelitis (EAE), an animal model of MS, microglia have been shown to be activated and have increased major histocompatibility complex expression (possible role as antigen-presenting cell), release of reactive oxygen species, release of inflammatory cytokines, and have been shown to be transformed to phagocytic cells.

Demyelination of the axons leads to a slowing or cessation of the transmission of nerve impulses and improperly functioning axons. Demyelination may occur as a result of inflammation. There is evidence of the involvement of cytokines in MS, including inflammatory cytokines such as tumor necrosis factors and IFN-γ. Activated T_{H1} cells in MS, can release inflammatory cytokines such as IL-1, IL-2, and IL-12. Inflammation and the action of inflammatory cytokines causes immune cells to target and act at the site of inflammation, with resultant damage to myelin as well as damage or death of oligodendrocytes. In the absence of sufficient numbers or sufficient activity of oligodendrocytes, repair of the myelin sheath and consequent restoration of full axonal function cannot occur.

Inflammation can also have the effect of impairing the myelin-producing oligodendrocytes. Whilst it has been widely accepted in recent years that oligodendrocyte loss is a significant factor in MS development and progression, supported by the fact that post mortem examination of the brain of MS sufferers has revealed that almost no oligodendrocytes persist in the middle of chronic MS lesions, the cause of such oligodendrocyte loss is less clear. The prevailing theory that inflammation is largely responsible for oligodendrocyte loss, as well as demyelination of the axons, is under reconsideration as a result of research on the brains of patients who have died in the very early stages of MS. There are indications that oligodendrocyte loss precedes inflammation (Prineas, John W., An Neurol 2004:55, February 23). There is evidence of increased TNF expression in inflammatory lesions of CNS including multiple sclerosis (Selmaj, et al., J. Clin. Invest. 1991; 87:949-954). In addition, some researchers have shown that TNF-induced death of adult oligodendrocytes is mediated by JNK.

The commonest form of MS initially manifests itself in a relapsing-remitting (RRMS) phase, in which the patient experiences relapses, during which old symptoms reappear and worsen, and new symptoms can appear. These are followed by periods of remission during which the patient fully or partially recovers. The disease normally progresses to a secondary progressive phase (SPMS), which is characterized by a gradual worsening of the symptoms with few if any remission periods. During SPMS, there is a substantial amount of neuronal cell injury and neuronal cell death, although inflammation plays a smaller and smaller role. It may be that the absence of oligodendrocytes contributes to this neuronal injury and death, since the axons are deprived of essential factors for their maintenance and growth normally supplied by oligodendrocytes, such as insulin-like growth factor-1 (IGF-1)—see Guiterrez-Ospina, G. et al, Neurosci Lett 2002 Jun. 14; 325(3):207-210, and Russell, J. W. et al, Neurobiol Dis 1999 October; 6(5):347-63.

Most current treatments for MS tend to concentrate on delaying the progression from RRMS to SPMS. There is currently no effective treatment for or prophylaxis against SPMS.

It is generally accepted that the processes of inflammation, demyelination, neuronal cell death, oligodendrocyte death, axonal deterioration and death, and perhaps others, all play a role in MS, its symptoms and its progression, although their relative importance is currently unclear. A biologically acceptable composition that effectively counteracts any one of these processes is a candidate for development as a treatment for MS. A composition that effectively counteracts two or more of these processes would be particularly desirable.

### BRIEF REFERENCE TO THE PRIOR ART

Since the underlying causes of MS remain incompletely understood, currently prescribed treatments concentrate on slowing down the progression of the disease or alleviating its symptoms. MS can manifest itself in a wide variety of symptoms, and no two patients manifest MS in exactly the same way. Moreover, MS has at least four main varieties (relapsing-remitting MS, secondary progressive MS, progressive-relapsing MS and primary progressive MS), some of which have sub-divisions. Not surprisingly, therefore, treatments for MS are many and varied, depending upon the symptoms manifested by the patient, and the type and stage of the patient's disorder. β-interferon-1a and β-interferon-1b are commonly prescribed, to combat the autoimmune component of MS by regulating aspects of the patient's immune system. Glatiramer acetate (COP-1, Copaxone) is another treatment believed to act by modifying the body's T-cell mediated immune response to myelin. Copaxone has recently been reported to have neuroprotective activity (Kreitman, R. R. et al., Mult. Scler, 2004; 10 (Suppl. 1):S81-S86). Both β-interferon and Copaxone are very expensive. Steroids such as methylprednisolone are sometimes prescribed to treat relapses in MS, but appear to be palliative and to have no effect on the overall progress of the disease.

Liposomes presenting exterior phosphatidylglycerol groups have been proposed for treating various inflammatory conditions including neuroinflammatory conditions such as Alzheimer's disease—see international patent application PCT/CA03/00065, international filing date Jan. 21, 2003.

### SUMMARY OF THE INVENTION

The present invention is based upon the discovery that phosphatidylglycerol (PG) carrying bodies when administered to a mammal exhibit a protective effect on neurons in addition to reducing inflammation in the brain. Accordingly, such PG carrying bodies are potentially useful in prophylaxis against the development, slowing down the progression, and/or alleviating the symptoms of multiple sclerosis MS, especially the phase of MS where neuronal cell death is a predominant factor, i.e. secondary progressive (SPMS).

Studies carried out in support of the present invention, as described herein, show that administration of phosphatidylglycerol-carrying bodies to rats reduces levels of certain cytokines (IL-1β, TNF-α, etc.) that, when elevated, result in inflammation within the brain and central nervous system, and also in some cases result in neuronal death. In other words, such PG-carrying bodies have anti-inflammatory effects within the brain.

In addition, such PG-carrying bodies exhibit neuroprotective effects, as evidenced by their ability to activate the phosphorylated extracellular regulated kinase (p-ERK) pathway, a cell survival pathway, and increase p-ERK in the brain of a mammal, their ability to inhibit dopaminergic neuronal death, and their ability to down-regulate expression of p-JNK, the active form of an enzyme involved in one of the apoptotic cell death pathways.

Thus in accordance with the present invention, an appropriate dosage of three-dimensional synthetic or semi-synthetic PG-presenting bodies is administered to a mammal showing or likely to show symptoms of MS. Such bodies have shapes and dimensions ranging from those resembling mammalian cells to shapes and dimensions approximating to apoptotic bodies produced by apoptosis of mammalian cells, and having phosphate-glycerol molecules on the surface thereof.

### BRIEF REFERENCE TO THE DRAWINGS

FIG. 1 is a graphical presentation of IL-1β measurements obtained from samples of rat brain treated and prepared as described in Example 1 below;

FIG. 2 is a similar graphical presentation of p-ERK measurements obtained from samples of rat brain treated and prepared as described in Example 2 below;

FIG. 3 is a graphical presentation of p-JNK measurements obtained from samples of rat brain treated and prepared as described in Example 2 below;

FIGS. 4 and 5 are graphical presentations of tissue necrosis factor-α (TNF-α) measurements obtained from samples of rat brain treated and prepared as described in Example 3 below.

FIG. 6 is a graphical presentation of the development of clinical symptoms of experimental autoimmune encephalomyelitis (EAE) in SJL mice.

FIG. 7 is a graphical representation of the effect of liposome treatment on the mean clinical score of severity of EAE on the early second phase of EAE (day 21-31) in SJL mice in comparison to vehicle control as described in Example 5 below.

FIG. 8 is a graphical representation of the effect of liposome treatment on the mean clinical score of severity of EAE on the late second phase of EAE (day 32-42) in SJL mice in comparison to vehicle control as described in Example 5 below.

### THE PREFERRED EMBODIMENTS

According to one embodiment of the invention, PG-carrying bodies may be administered as liposomes comprising phosphatidylglycerol on their surfaces. Preferably, PG-carrying bodies have diameters from about 20 nanometers to about 500 micrometers (0.02-500 microns).

According to another feature, PG-carrying bodies are administered in a unit dosage amount of from about 500 to about 5×10¹² bodies per unit dosage. Such administration may be by any of a number of routes, including, without limitation, intramuscular administration.

PG-carrying bodies, as described above and herein, may be used in the preparation of medicaments for decelerating the progression, treating or preventing MS in mammalian subjects.

These and other objects and features of the invention will become more fully apparent when the following detailed description of the invention is read in conjunction with the accompanying drawings.

All publications cited herein are herein incorporated by reference in their entirety to the same extent as if each individual publication was specifically and individually incorporated by reference in its entirety.

Definitions

The terms “liposomes” and “lipid vesicles” refer to sealed membrane sacs, having diameters in the micron or sub-micron range, the walls of which consist of layers, typically bilayers, of suitable, membrane-forming amphiphiles. They normally contain an aqueous medium.

The term “pharmaceutically acceptable” has a meaning that is similar to the meaning of the term “biocompatible.” As used in relation to “pharmaceutically acceptable bodies” herein, it refers to bodies of the invention comprised of one or more materials which are suitable for administration to a mammal, preferably a human, in vivo, according to the method of administration specified (e.g., intramuscular, intravenous, subcutaneous, topical, oral, and the like).

The term “phosphate choline” refers to the group —O—P(═O)(OH)—O—CH2-CH2-N+(CH3)3, which can be attached to lipids to form “phosphatidylcholine” (PC) as shown in the following structure: 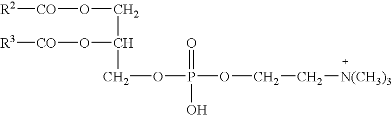
and salts thereof, wherein R2 and R3 are independently selected from Cl-C24 hydrocarbon chains, saturated or unsaturated, straight chain or containing a limited amount of branching wherein at least one chain has from 10-24 carbon atoms. The term “phosphate-glycerol-carrying bodies” refers to biocompatible, pharmaceutically-acceptable, three-dimensional bodies having on their surfaces phosphate-glycerol groups or groups that can be converted to phosphate-glycerol groups, as described herein.

A “phosphate-glycerol group” is a group having the general structure: O—P(═O)(OH)—O—CH₂CH(OH)CH₂OH, and derivatives thereof, including, but not limited to groups in which the negatively charged oxygen of the phosphate group is converted to a phosphate ester group (e.g., L-OP(O)(OR′)(OR″), where L is the remainder of the phosphate-glycerol group, R′ is —CH₂CH(OH)CH₂OH and R″ is alkyl of from 1 to 4 carbon atoms, or a hydroxyl substituted alkyl of from 2 to 4 carbon atoms, and 1 to 3 hydroxyl groups provided that R″ is more readily hydrolyzed in vivo than the R′ group; to a diphosphate group including diphosphate esters (e.g., L-OP(O)(OR′)OP(O)(OR″)₂ wherein L and R′ are as defined above and each R″ is independently hydrogen, alkyl of from 1 to 4 carbon atoms, or a hydroxyl substituted alkyl of from 2 to 4 carbon atoms and 1 to 3 hydroxyl groups, provided that the second phosphate [—P(O)(OR″)₂] is more readily hydrolyzed in vivo than the R′ group; or to a triphosphate group including triphosphate esters (e.g., L-OP(O)(OR′)OP(O)(OR″)OP(O)(OR″)₂ wherein L and R′ are defined as above and each R″ is independently hydrogen, alkyl of from 1 to 4 carbon atoms, or a hydroxyl substituted alkyl of from 2 to 4 carbon atoms and 1 to 3 hydroxyl groups provided that the second and third phosphate groups are more readily hydrolyzed in vivo than the R′ group; and the like. Such synthetically altered phosphate-glycerol groups are capable of expressing phosphate-glycerol in vivo and, accordingly, such altered groups are phosphate-glycerol convertible groups within the scope of the invention. A specific example of a phosphate-glycerol group is the compound phosphatidylglycerol (PG), further defined herein.

“Phosphatidylglycerol” is also abbreviated herein as “PG.” This term is intended to cover phospholipids carrying a phosphate-glycerol group with a wide range of at least one fatty acid chain provided that the resulting PG entity can participate as a structural component of a liposome. Chemically, PG has a phosphate-glycerol group is and a pair of similar, but different fatty acid side chains. Preferably, such PG compounds can be represented by the Formula I: 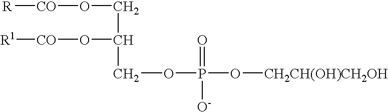
where R and R¹ are independently selected from C₁-C₂₄ hydrocarbon chains, saturated or unsaturated, straight chain or containing a limited amount of branching wherein at least one chain has from 10 to 24 carbon atoms. R and R¹ can be varied to include two or one lipid chain(s), which can be the same or different, provided they fulfill the structural function. As mentioned above, the fatty acid side chains may be from about 10 to about 24 carbon atoms in length, saturated, mono-unsaturated or polyunsaturated, straight-chain or with a limited amount of branching. Laurate (C12), myristate (C14, palmitate (C16), stearate (C18), arachidate (C20), behenate (C22) and lignocerate (C24) are examples of useful saturated fatty acid side chains for the PG for use in the present invention. Palmitoleate (C15), oleate (C18) are examples of suitable mono-unsaturated fatty acid side chains. Linoleate (C18), linolenate (C18) and arachidonate (C20) are examples of suitable poly-unsaturated fatty acid side chains for use in PG in the compositions of the present invention. Phospholipids with a single such fatty acid side chain, also useful in the present invention, are known as lysophospholipids.

The term PG also includes dimeric forms of PG, namely cardiolipin, but other dimers of Formula I are also suitable. Preferably, such dimers are not synthetically cross-linked with a synthetic cross-linking agent, such as maleimide but rather are cross-linked by removal of a glycerol unit as described by Lehninger, Biochemistry and depicted in the reaction below: 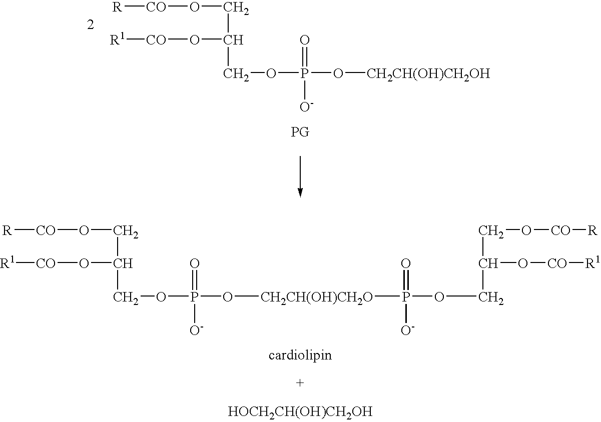
where each R and R¹ are independently as defined above.

Purified forms of phosphatidylglycerol are commercially available, for example, from Sigma-Aldrich (St. Louis, Mo.). Alternatively, PG can be produced, for example, by treating the naturally occurring dimeric form of phosphatidylglycerol, cardiolipin, with phospholipase D. It can also be prepared by enzymatic synthesis from phosphatidyl choline using phospholipase D (see, for example, U.S. Pat. No. 5,188,951 Tremblay et al., incorporated herein by reference).

“PG-carrying bodies” are three-dimensional bodies, as described above, that have surface PG molecules. By way of example, PG can form the membrane of a liposome, either as the sole constituent of the membrane or as a major or minor component thereof, with other phospholipids and/or membrane forming materials. In the context of the present invention, “three-dimensional bodies” refer to biocompatible synthetic or semi-synthetic entities, including but not limited to liposomes, solid beads, hollow beads, filled beads, particles, granules and microspheres of biocompatible materials, natural or synthetic, as commonly used in the pharmaceutical industry. Liposomes may be formed of lipids, including phosphatidylglycerol (PG). Beads may be solid or hollow, or filled with a biocompatible material. Such bodies have shapes that are typically, but not exclusively spheroidal, cylindrical, ellipsoidal, including oblate and prolate spheroidal, serpentine, reniform and the like, and have sizes ranging from 20 nm to 500 μm, preferably measured along the longest axis.

Phosphate-Glycerol-Carrying Bodies

This section describes various embodiments of phosphate-glycerol-carrying bodies contemplated by the present invention, including specific embodiments thereof. With the guidance provided herein, persons having requisite skill in the art will readily understand how to make and use phosphate-glycerol-carrying bodies in accordance with the present invention.

In the context of the present invention, phosphate-glycerol-carrying bodies refer to biocompatible, pharmaceutically-acceptable, three-dimensional bodies having on their surfaces phosphate-glycerol groups or groups that can be converted to phosphate-glycerol groups, as described herein.

Phosphate-Glycerol Groups

According to a general feature of the invention, phosphate-glycerol groups useful in the present invention have the general structure:
—O—P(═O)(OH)—O—CH₂CH(OH)CH₂OH

Such phosphate-glycerol groups include synthetically altered versions of the phosphate-glycerol group shown above, and may include all, part of or a modified version of the original phosphate-glycerol group.

Preferably the fatty acid side chains of the chosen PG will be suitable for formation of liposomes, and incorporate into the lipid membrane(s) forming such liposomes, as described in more detail below.

PG groups of the present invention, including dimers thereof, are believed to act as ligands, binding to specific sites on a protein or other molecule (“PG receptor”) and, accordingly, PG (or derivatives or dimeric forms thereof) are sometimes referred to herein as a “ligand” or a “binding group.” Such binding is believed to take place through the phosphate-glycerol group

—O—P(═O)(OH)—O—CH₂CH(OH)CH₂OH, which is sometimes referred to herein as the “head group,” “active group,” or “binding group,” while the fatty acid side chain(s) are believed to stabilize the group and/or, in the case of liposomal preparations, form the outer lipid layer or bilayer of the liposome. More generally, again without being limited to any particular theory, it is believed that phosphate-glycerol groups, including PG are capable of interacting with one or more receptors in the brain and that such interactions may provide positive effects on synaptic transmission, and, by extension, symptoms of MS, as described herein.

Formation of Phosphate-Glycerol Carrying Bodies

Phosphate-glycerol carrying bodies are three-dimensional bodies that have surface phosphate-glycerol molecules. This section will describe general and exemplary phosphate-glycerol carrying bodies suitable for use in the present invention.

Generally, phosphate-glycerol carrying bodies of the present invention carry phosphate-glycerol molecules on their exterior surfaces to facilitate in vivo interaction of the binding groups.

Three-dimensional bodies are preferably formed to be of a size or sizes suitable for administration to a living subject, preferably by injection; hence such bodies will preferably be in the range of 20 nm to 500 μm, more preferably from 20 to 1000 nm (0.02-1 micron), more preferably 20 to 500 nm (0.02-0.5 micron), and still more preferably 20-200 nm in diameter, where the diameter of the body is determined on its longest axis, in the case of non-spherical bodies. Suitable sizes are generally in accordance with blood cell sizes. While bodies of the invention have shapes that are typically, but not exclusively spheroidal, they can alternatively be cylindrical, ellipsoidal, including oblate and prolate spheroidal, serpentine, reniform in shape, or the like.

Suitable forms of bodies for use in the compositions of the present invention include, without limitation, particles, granules, microspheres or beads of biocompatible materials, natural or synthetic, such as polyethylene glycol, polyvinylpyrrolidone, polystyrene, and the like; polysaccharides such as hydroxethyl starch, hydroxyethylcellulose, agarose and the like; as are commonly used in the pharmaceutical industry. Preferably, such materials will have side-chains or moieties suitable for derivatization, so that a phosphate-glycerol group, such as PG, may be attached thereto, preferably by covalent bonding. Bodies of the invention may be solid or hollow, or filled with biocompatible material. They are modified as required so that they carry phosphate-glycerol molecules, such as PG on their surfaces. Methods for attaching phosphate-glycerol in general, and PG in particular, to a variety of substrates are known in the art.

In addition to the various bodies listed above, the liposome is a particularly useful form of body for use in the present invention. Liposomes are microscopic vesicles composed of amphiphilic molecules forming a monolayer or bilayer surrounding a central chamber, which may be fluid-filled. Amphiphilic molecules (also referred to as “amphiphiles”), are molecules that have a polar water-soluble group attached to a water-insoluble (lipophilic) hydrocarbon chain, such that a matrix of such molecules will typically form defined polar and apolar regions. Amphiphiles include naturally occurring lipids such as PG, phosphatidylserine, phosphatidylethanolamine, phosphatidylinositol, phosphatidylcholine, cholesterol, cardiolipin, ceramides and sphingomyelin, used alone or in admixture with one another. They can also be synthetic compounds such as polyoxyethylene alkyl ethers, polyoxyethylene alkyl esters and saccharosediesters. Thus a preferred embodiment of this invention provides liposomal bodies which expose or can be treated or induced to expose, on their surfaces, one or more phosphatidylglycerol groups to act as binding groups. Such lipids should comprise from 10%-100% of the liposome, with the balance being an inactive constituent, e.g. phosphatidylcholine PC, or one which acts through a different mechanism, e.g. phosphatidylserine PS, or mixtures of such. Inactive co-constituents such as PC are preferred. Those used in the present invention have at least 10% by weight PG content.

Preferably, for use in forming liposomes, the amphiphilic molecules will include one or more forms of phospholipids of different headgroups (e.g., phosphatidylglycerol, phosphatidylserine, phosphatidylcholine) and having a variety of fatty acid side chains, as described above, as well as other lipophilic molecules, such as cholesterol, sphingolipids and sterols.

In accordance with the present invention, phosphatidylglycerol (PG) will constitute the major portion or the entire portion of the liposome layer(s) or wall(s), oriented so that the phosphate-glycerol group portion thereof is presented exteriorly, as described above, while the fatty acid side chains form the structural wall. When, as in the present invention, the bilayer includes phospholipids, the resulting membrane is usually referred to as a “phospholipid bilayer,” regardless of the presence of non-phospholipid components therein.

Liposomes of the invention are typically formed from phospholipid bilayers or a plurality of concentric phospholipid bilayers which enclose aqueous phases. In some cases, the walls of the liposomes may be single layered; however, such liposomes (termed “single unilamellar vesicles”) are generally much smaller (diameters less than about 70 nm) than those formed of bilayers, as described below. Liposomes formed in accordance with the present invention are designed to be biocompatible, biodegradable and non-toxic. Liposomes of this type are used in a number of pharmaceutical preparations currently on the market, typically carrying active drug molecules in their aqueous inner core regions. In the present invention, however, the liposomes are not filled with pharmaceutical preparation. The liposomes are active themselves, not acting as drug carrier.

Preferred PG-carrying liposomes of the present invention are constituted to the extent of at least 10% by weight of phosphatidyl glycerol, the balance being phosphatidylcholine (PC) or other such biologically acceptable phospholipids(s), preferably at least 50%, more preferably from 60-100% and most preferably from 70-90%, with the single most preferred embodiment being about 75% by weight of PG.

Mixtures of PG liposomes with inactive liposomes and/or with liposomes of phospholipids acting through a different mechanism can also be used, provided that the total amount of PG remains above the minimum of about 10% and preferably above 60% in the total mixture. Such liposomes are prepared from mixtures of the appropriate amounts of phospholipids as starting materials, by known methods. According to a preferred feature of the invention, PG-carrying bodies comprise less than 50%, preferably less than 40%, still preferably less than 25% and even still preferably less than 10% phosphatidyl choline.

The present invention contemplates the use, as PG-carrying bodies, not only of those liposomes having PG as a membrane constituent, but also liposomes having non-PG membrane substituents that carry on their external surface molecules of phosphate-glycerol, either as monomers or oligomers (as distinguished from phosphatidylglycerol), e.g., chemically attached by chemical modification of the liposome surface of the body, such as the surface of the liposome, making the phosphate-glycerol groups available for subsequent interaction. Because of the inclusion of phosphate-glycerol on the surface of such molecules, they are included within the definition of PG-carrying bodies.

Liposomes may be prepared by a variety of techniques known in the art, such as those detailed in Szoka et al. (Ann. Rev. Biophys. Bioeng. 9:467 (1980)). Depending on the method used for forming the liposomes, as well as any after-formation processing, liposomes may be formed in a variety of sizes and configurations. Methods of preparing liposomes of the appropriate size are known in the art and do not form part of this invention. Reference may be made to various textbooks and literature articles on the subject, for example, the review article by Yechezkel Barenholz and Daan J. A. Chromeline, and literature cited therein, for example New, R. C. (1990), and Nassander, U. K., et al. (1990), and Barenholz, Y and Lichtenberg, D., Liposomes: preparation, characterization, and preservation. Methods Biochem Anal. 1988, 33:337-462.

Multilamellar vesicles (MLV's) can be formed by simple lipid-film hydration techniques according to methods known in the art. In this procedure, a mixture of liposome-forming lipids is dissolved in a suitable organic solvent. The mixture is evaporated in a vessel to form a thin film on the inner surface of the vessel, to which an aqueous medium is then added. The lipid film hydrates to form MLVs, typically with sizes between about 100-1000 nm (0.1 to 10 microns) in diameter.

A related, reverse evaporation phase (REV) technique can also be used to form unilamellar liposomes in the micron diameter size range. The REV technique involves dissolving the selected lipid components, in an organic solvent, such as diethyl ether, in a glass boiling tube and rapidly injecting an aqueous solution, optionally containing a drug solution to be carried in the interior of the liposome, into the tube, through a small gauge passage, such as a 23-gauge hypodermic needle. The tube is then sealed and sonicated in a bath sonicator. The contents of the tube are alternately evaporated under vacuum and vigorously mixed, to form a final liposomal suspension.

The diameters of the PG-carrying liposomes of the preferred embodiment of this invention range from about 20 nm to 500 μm, more preferably from 20 nm to about 1000 nm, more preferably from about 20 nm to about 500 nm, and most preferably from about 20 nm to about 200 nm. Such preferred diameters will correspond to the diameters of mammalian apoptotic bodies, such as may be apprised from the art.

One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the median size of liposomes produced by extrusion through that membrane, particularly where the preparation is extruded two or more times through the same membrane. This method of liposome sizing is used in preparing homogeneous-size REV and MLV compositions. U.S. Pat. Nos. 4,737,323 and 4,927,637, incorporated herein by reference, describe methods for producing a suspension of liposomes having uniform sizes in the range of 0.1-0.4 μm (100-400 nm) using as a starting material liposomes having diameters in the range of 1 μm. Homogenization methods are also useful for down-sizing liposomes to sizes of 100 nm or less (Martin, F. J. (1990) In: Specialized Drug Delivery Systems—Manufacturing and Production Technology, P. Tyle (ed.) Marcel Dekker, New York, pp. 267-316.). Another way to reduce liposomal size is by application of high pressures to the liposomal preparation, as in a French Press.

Liposomes can be prepared to have substantially homogeneous sizes of single, bi-layer vesicles in a selected size range between about 0.07 and 0.2 microns (70-200 nm) in diameter, according to methods known in the art. In particular, liposomes in this size range are readily able to extravasate through blood vessel epithelial cells into surrounding tissues. A further advantage is that they can be sterilized by simple filtration methods known in the art. Whilst a preferred embodiment of PG-carrying bodies for use in the present invention is liposomes with PG presented on the external surface thereof, it is understood that the PG-carrying body is not limited to a liposomal structure, as mentioned above.

Dosages and Modes of Administration

The phosphate-glycerol-carrying bodies of the invention may be administered to the patient by any suitable route of administration, including oral, nasal, topical, rectal, intravenous, subcutaneous and intramuscularly. At present, intramuscular administration is preferred, especially in conjunction with PG-liposomes.

The PG-carrying bodies may be suspended in a pharmaceutically acceptable carrier, such as physiological sterile saline, sterile water, pyrogen-free water, isotonic saline, and phosphate buffer solutions, as well as other non-toxic compatible substances used in pharmaceutical formulations. Preferably, PG-carrying bodies are constituted into a liquid suspension in a biocompatible liquid such as physiological saline and administered to the patient in any appropriate route which introduces it to the immune system, such as intra-arterially, intravenously, or most preferably intramuscularly or subcutaneously.

A preferred manner of administering the PG-carrying bodies to the patient is a course of injections, administered daily, several times per week, weekly or monthly to the patient, over a period ranging from a week to several months. The frequency and duration of the course of the administration is likely to vary from patient to patient, and according to the condition being treated, its severity, and whether the treatment is intended as prophylactic, therapeutic or curative. One currently preferred dosage schedule is a daily injection for six successive days, followed by a booster injection monthly. It is within routine testing to extrapolate such dosing regimens to other mammalian species. The quantities of PG-carrying bodies to be administered will vary depending on the identity and characteristics of the patient. It is important that the effective amount of PG-bodies is non-toxic to the patient.

The most effective amounts are unexpectedly small. When using intra-arterial, intravenous, subcutaneous or intramuscular administration of a liquid suspension of PG-carrying bodies, it is preferred to administer, for each dose, from about 0.1-50 ml of liquid, containing an amount of PG-carrying bodies generally equivalent to 10%-1000% of the number of leukocytes normally found in an equivalent volume of whole blood or the number of apoptotic bodies that can be generated from them. Generally, the number of PG-carrying bodies administered per delivery to a human patient is in the range from about 500 to about 2.5×10¹² (about 260 micrograms by weight at the highest end of the range), preferably from about 5,000 to about 500,000,000, more preferably from about 10,000 to about 10,000,000, and most preferably from about 200,000 to about 2,000,000.

According to one feature of the invention, the number of such bodies administered to an injection site for each administration is believed to be a more meaningful quantification than the number or weight of PG-carrying bodies per unit of patient body weight. Thus, it is contemplated that effective amounts or numbers of PG-carrying bodies for small animal use may not directly translate into effective amounts for larger mammals on a weight ratio basis. The person skilled in the art could readily extrapolate from the data and other information contained herein to arrive at appropriate dosing for other mammals.

It is contemplated that the PG-carrying bodies may be freeze-dried or lyophilized to a form which may be later re-suspended for administration. This invention therefore also includes a kit of parts comprising lyophilized or freeze-dried PG-carrying bodies and a pharmaceutically acceptable carrier, such as physiological sterile saline, sterile water, pyrogen-free water, isotonic saline, and phosphate buffer solutions, as well as other non-toxic compatible substances used in pharmaceutical formulations. Such a kit may optionally provide injection or administration means for administering the composition to a subject.

The invention is further described in the following illustrative examples.

### EXAMPLE 1

Aged rats have been shown to have increased concentrations of the pro-inflammatory cytokine IL-1β in the hippocampus in comparison to young rats. Unilamellar liposomes of 100±20 nm in average diameter were prepared by known extrusion methods and were composed of 75% 1-palmitoyl-2-oleoly-sn-glycero-3-phosphoglycerol (POPG) and 25% 1-palmitoyl-2-oleoyl-sn-glycero-3-phosphocholine (POPC) by weight. A stock suspension of the liposomes containing about 2.9×10¹⁴ liposomes per ml was diluted with phosphate buffered saline (PBS) to give an injection suspension containing about 1.2×10⁷ liposomes per ml. This was then used to inject into rats to determine the effect on IL-1β expression in young and aged rats. For these experiments, male Wistar rats (BioResources Unit, Trinity College, Dublin), aged 4 months and 24 months, were used.

The animals were assigned to one of four groups, 8 animals in each group to be treated as follows:

| | |
|---|---|
| Group A (young rats) | saline |
| Group B (young rats) | liposome preparation |
| Group C (aged rats) | saline |
| Group D (aged rats) | liposome preparation |

150 μl of saline or liposome preparation was injected via intramuscular injection on days-14, -13, and -1. Groups B and D received a total of 5,400,000 liposomes (1,800,000 liposomes per injection). The tissue preparation procedure was carried out on day 0.

Rats were anaesthetized by IP injection of urethane (1.5 g/kg).

Rats were sacrificed by decapitation and the brain rapidly removed. The hippocampus was dissected free from the whole brain. Slices (350×350 micrometers) were prepared using a Mcllwain tissue chopper and stored in Krebs buffer containing calcium chloride (1.13 millimolar) and 10% DMSO at −80° C. until required for analysis, generally following methods described in Haan, E. A. and Bowen, D. M. (1981), J. Neurochem. 37, 243-246.

The concentration of IL-1β was assessed in hippocampal homogenates, according to methods known in the art. Analysis was carried out by ELISA (R&D systems, U.K.). Hippocampal slices were thawed, and rinsed three times in ice cold Krebs solution and homogenized in ice cold Krebs solution. Protein concentrations in homogenates were equalized and triplicate aliquots (100 microliter) were used for ELISA. Biomarker-specific antibody-coated 96-well plates were incubated overnight at room temperature, washed several times with PBS containing 0.05% Tween 20, blocked for one hour at room temperature with blocking buffer (PBS, pH7.3; 5% sucrose; 1% BSA; 0.05% NaN₃), and incubated with standards or samples for two hours at room temperature. Wells were washed with PBS, incubated with secondary antibody for two hours at room temperature, washed again and incubated in horseradish peroxidase-conjugated streptavidin (1:200 dilution in PBS containing 1% BSA) for 20 minutes at room temperature. Substrate solution (1:1 mixture of hydrogen peroxide and tetramethylbenzidine) was added, incubation continued at room temperature in the dark for 30 minutes and reactions stopped using 1M sulfuric acid. Absorbance was read at 450 nm, the values were corrected for protein, and expressed as picograms per milligram protein.

The results are presented as a bar graph in FIG. 1. FIG. 1 shows that, IL-1β, in picograms per mg plotted along the vertical axis, which is increased in the hippocampus of aged rats, is significantly reduced (p<0.05 by ANOVA) by the PG liposome treatment. The down regulation of IL-1β demonstrates the anti-inflammatory effects of the compositions in the brain, a further indicator of potential use as a prophylaxis or treatment in MS since this inflammatory cytokine is likely involved in inflammation in MS. The results are the means of measurements on 8 animals in each group.

### EXAMPLE 2

### Assessment of JNK and ERK Activity

The phosphorylated forms of JNK (p-JNK) and ERK (p-ERK) were assessed in homogenate obtained from the hippocampus of animals treated as described in Example 1. p-ERK is an enzyme associated with cell survival. It has cell protective effects, and is associated with cell differentiation and cell growth. An upregulation of its expression is an indicator of a cell protective, and specifically in the present case, of a neuronal protective effect. The enzyme p-JNK, on the other hand, is a stress activated protein kinase that has been shown to trigger cell death in several cell types, including hippocampus. Its downregulation is indicative of a cell protective effect. It is known that age is associated with an increase in JNK phosphorylation and a decrease in pERK.

Tissue samples prepared from the hippocampus taken from the experiment in Example 1, were equalized for protein concentration, and aliquots (10 μl, 1 mg/ml) were added to sample buffer (5 μl; Tris-HCl, 0.5 mM, pH6.8; glycerol 10%; SDS, 10%; β-mercaptoethanol, 5%; bromophenol blue, 0.05% w/v), boiled for 5 minutes and loaded onto gels (12% SDS for JNK, 10% SDS for ERK). Proteins were separated by application of 30 mA constant current for 25-30 minutes transferred onto nitrocellulose strips (225 mA for 75 min) and immunoblotted with the appropriate antibody. To assess expression of p-JNK, nitrocellulose strips were incubated overnight at 4° C. in the presence of an antibody that specifically targets p-JNK (Santa Cruz, USA; diluted 1:200) in Tris buffered saline—Tween (TBS-T; 0.1% Tween-20) to which 0.1% BSA was added. Nitrocellulose strips were washed and incubated for 2 hours at room temperature with secondary antibody (peroxidase-linked anti-mouse IgG; 1:300 dilution Sigma UK), diluted in TBS-T containing 0.1% BSA. To assess expression of p-ERK, nitrocellulose strips were incubated overnight at 4° C. in the presence of an antibody that specifically targets p-ERK (Santa Cruz, USA, diluted 1:700) in phosphate buffered saline Tween and 6% dried milk, and incubated for 2 hours at room temperature with secondary antibody (anti-mouse 1gG; 1:1000 dilution) in PBS-Tween and 6% dried milk.

Protein complexes were visualized using Super Signal West Dura Extended Duration Substrate (Pierce, USA). Immunoblots were exposed to film for 1 to 10 s and processed using a Fuji x-ray processor. Protein bands were quantitated by densitometric analysis using Gel works software package (Gelworks ID, version 2.51; UVP Limited, UK), to provide a single value (in arbitrary units) representing the density of such blot.

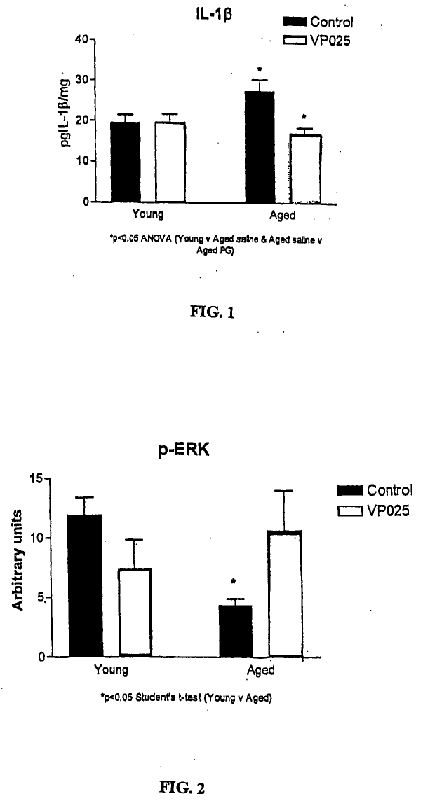

FIG. 2 of the accompanying drawings, on which p-ERK amount in arbitrary units is plotted as vertical axis, shows that treatment of the animals with PG liposomes as described above reversed the age-related decrease in the activation of p-ERK (p<0.05, Student's t-test, young vs. aged).

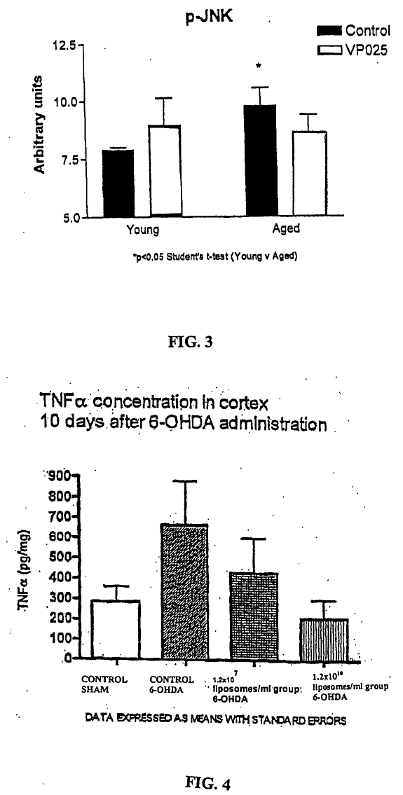

FIG. 3 of the accompanying drawings, on which p-JNK amount in arbitrary units is plotted as vertical axis show that treatment of the animals with PG liposomes as described above abrogated the age-related increase in JNK phosphorylation (p<0.05, Student's t-test, young vs. aged).

The results are the means of 8 animals. The decrease in activation of JNK, coupled with the increase in activation of p-ERK, indicates potential use as a prophylaxis or treatment in MS given the possible involvement of JNK in oligodendrocyte death.

### EXAMPLE 3

The chemotoxin 6—hydroxydopamine (6-OHDA), when introduced into the cell bodies and nerve fibers of dopaminergic neurons, exerts potent cytotoxic effects via inhibition of mitochondrial complexes. Unilateral stereotaxic injection of 6-OHDA into the substantia nigra pars compacta (SNpc), the striatum or the medial forebrain bundle (MFB; the nigrostriatal fibre tract) of rodents produces a dramatic dropout of dopaminergic neurons in the SNpc accompanied by a marked reduction of dopaminergic terminals in the striatum. Introduction of 6-OHDA into one hemisphere of the brain results in destruction of dopaminergic neurons in the SNpc in that hemisphere, leaving the SNpc in the other hemisphere intact. This imbalance between hemispheres causes a marked asymmetry in the motor behavior of the animals 4-7 days post 6-OHDA lesion. Intraperitoneal administration of the dopaminomimetic drug D-amphetamine creates a dopamine imbalance that favors the non-lesioned hemisphere, and animals display a rotatory behaviour towards the lesioned hemisphere.

Experimental Procedures

Groups of male Sprague-Dawley rats (225-250 grams, Biological Service Unit, University College Cork) were used in these experiments. Animals were maintained in the temperature and humidity controlled environment under the 12-hour light schedule with food and water available ad libitum. The rats were caged in groups of six during the presurgical period and then individually housed following the lesion. All animal procedures strictly adhered to local and national guidelines.

All rats were treated intramuscularly with phosphatidyl glycerol—containing liposomes as described in Example 1; 150 μl of a 1.2×10⁷ liposomes/ml suspension in phosphate-buffered saline, 150 μl of a 1.2×10¹⁰ liposomes/ml suspension in phosphate-buffered saline, or saline, 14 days, 13 days and 1 day before unilateral lesioning of the MFB with 6-OHDA; (8 μg/4 μl). Administration of either drug or control was alternated between the left and right hind limbs on alternate days in an attempt to minimize local muscle injury.

Two weeks after the initial exposure to either vehicle or liposomes, rats were anaesthetized with a 1:1 mixture of xylazine hydrochloride (Vetoquinol UK Ltd) and ketamine hydrochloride (Chassot, Dublin, Ireland) with 1.50 ml of each compound dissolved in 7 ml of PBS. An injection volume of 0.2 ml/100 g body weight provided adequate anaesthesia. The animals were subjected to a surgical procedure whereby a small burr hole was drilled in the skull at the following coordinates: AP-2.2 mm, ML+1.5 mm from bregma. A 10 μl Hamilton syringe partially filled with 6-OHDA hydrobromide (Sigma, UK) was then slowly lowered into the MFB (7.8 mm ventral from brain surface). Once the tip of the needle was in place the surrounding brain tissue was allowed sufficient time (˜5 minutes) to reform around the needle before infusion of the neurotoxin. The 6-OHDA was then slowly infused (0.5 μl/min) at a concentration of 2 μg/μl (free base) and the needle left in place to allow for complete diffusion of the 6-OHDA into the surrounding brain tissue. The needle was then slowly withdrawn and the animal sutured closed before receiving post-operative care until it recovered fully for the anaesthesia.

Sham surgery groups received the exact same surgical protocol with the notable exception of 4 μl of saline rather than 6-OHDA.

The animals were sacrificed at predetermined time points by decapitation and their brains rapidly removed. Cortical tissue from both hemispheres was microdissected out on ice and cross-chopped into slices (350×350 μm) using a Mcllwain tissue chopper. Brain sections were placed into eppendorf tubes containing Krebs buffer with CaCl₂ (1.13 mM). The tissue was washed 3 times in Krebs buffer before being placed in a Krebs-Dimethyl Sulphoxide (10%) solution and stored at −80° C. as described by Haan and Bowen, 1981, J. Neurochem. 37, 243-246, until required for analysis.

TNF-α concentration in homogenate prepared from cortical tissue was analysed by enzyme-linked immunosorbent assay (ELISA; DuoSet; R&D Systems). Cortical slices were thawed, and rinsed three times in ice-cold Krebs solution and homogenized in ice-cold Krebs solution. Protein concentrations in homogenates were equalized and triplicate aliquots (100 μl) were used for ELISA. Antibody-coated (4.0 μg/ml mouse anti-rat TNF-α diluted in PBS, pH 7.3) 96-well plates were incubated overnight at 4° C., washed thoroughly with PBS containing 0.05% Tween 20, blocked for 1 h with 300 μl of blocking buffer (PBS, pH 7.3, with 1% bovine serum albumin), and incubated with standards (100 μl; 0-4000 pg/ml) or samples for 2 hours at room temperature. Samples were incubated with secondary antibody (100 ng/ml biotinylated goat anti-rat TNF-α in PBS containing 1% bovine serum albumin) for 2 h at room temperature. ELISA plates were then washed and incubated in detection agent (100 μl; horseradish peroxidase-conjugated streptavidin; 1:200 dilution in PBS contiaing 1% bovine serum albumin) in the dark for 20 min at room temperature. Substrate solution (1:1 mixture of H₂O₂ and tetramethylbenzidine; R&D Systems) was added, incubation continued at room temperature in the dark for 30 min and the reaction stopped using 1 M H₂SO₄. Absorbance was read at 450 nm using a Sunrise microplate reader; values were corrected for protein in the case of homogenates and expressed as pg/mg protein.

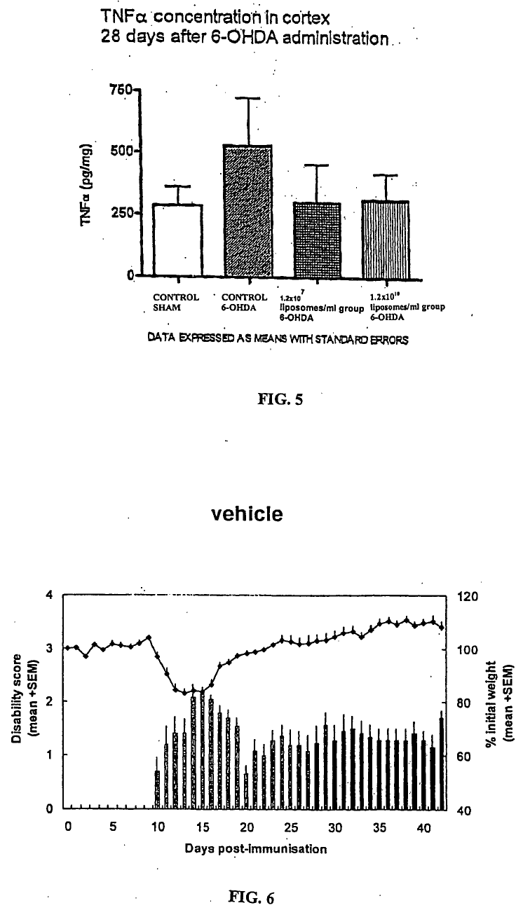

The results are presented graphically on the accompanying FIGS. 4 and 5. TNF-α is shown to be reduced in the cortex of the animals treated according to the invention, after 10 days (FIG. 4) and after 28 days (FIG. 5) from 6-OHDA administration, substantially down to control (sham treated) levels. This TNF-α down regulation effect is an indicator of anti-inflammatory effects of the compositions in the brain, a further indicator of potential use as a prophylaxis or treatment in MS. The results are the means of 10 animals in each group.

### EXAMPLE 4

The protective effect of the compositions used in accordance with the present invention was further demonstrated by showing maintenance of dopaminergic neurons in the rat brain following the 6-OHDA treatment described above.

Immunocytochemical assessment of tyrosine hydroxylase (TH) expression, a marker of dopaminergic neurons, was carried out on brain tissue taken from treated rats as described in Example 3.

A subsection of rats was terminally anaesthetised with Euthatal and transcardially perfused with 4% paraformaldehyde. The brains were removed, post-fixed in 4% paraformaldehyde and cryoprotected in 30% sucrose solution. Coronal sections throughout the entire area of the SNpc were cut at 15 μm thickness using a cryostat, and then the sections were mounted on slides and stained immunocytochemically for TH and CD11b (OX-42—an activated microglial marker). Sections were washed in 10 mM PBS before non-specific binding sites were blocked with 3% normal goat serum in 1% Triton X 100 in PBS overnight at 4° C. Sections were incubated overnight at 4° C. with either polyclonal rabbit IgG anti-TH (1:100; Chemicon) or mouse monoclonal IgG anti-CD11b (OX-24; 1:100; Serotec). Sections were washed in PBS three times prior to incubation for 90 minutes in the dark with a 1:50 dilution of fluorescein isothiocyanate (FITC)-labelled goat anti-rabbit IgG (for TH staining; Sigma, UK) or goat anti-mouse FITC (for OX-42 staining; Sigma, UK). Sections were then washed a further three times in PBS before counterstaining with propidium iodide (for TH; Sigma, UK). For double-labelling analysis of TH and OX-42, slides were incubated in goat anti-rabbit tetramethylrhodamine isothiocyanate (TRITC; Sigma, UK) for TH or goat anti-mouse FITC (for OX-42 staining; Sigma, UK) in the dark for 90 minutes (1:50 dilution). Slides were coverslipped, mounted with an aqueous mounting medium (Vector Laboratories) and viewed under an Olympus Provis fluorescent microscope with an Olympus DP50 digital camera. Photomicrographs were taken at 10×, 20× and 40× magnifications.

Visual observation of the photomicrographs showed that the fluorescence from TH, indicative of dopaminergic neuronal viability, in the SNpc of rats which had received 6-OHDA lesion following pre-treatment with PG liposomes, was substantially equivalent to that of control, sham-treated animals which had received no 6-OHDA treatment. In contrast, TH fluorescence in SNpc of animals that had received the 6-OHDA treatment but no pre-treatment with PG liposomes was much less intense. Photomicrographs taken at Day 4, Day 10 and Day 28 following the 6-OHDA treatment showed a consistent pattern. A neuronal protective effect for PG liposomes is apparent.

The fluorescence from double labelling immunohistochemistry for OX-42 and TH was similarly recorded on photomicrographs. Visual inspection of these revealed that OX-42 fluorescence in SNpc from the animals that had been pre-treated with PG liposomes and then administered 6-OHDA was substantially equivalent to that from untreated animals, and substantially lower than that from 6-OHDA treated animals which had not received pre-treatment with PG liposomes. This indicates that microglial activation caused by the 6-OHDA lesion is being counteracted by the pre-treatment with PG liposomes.

### EXAMPLE 5

Experimental autoimmune encephalomyelitis (EAE) is a generally accepted animal model of MS, and is used by researchers worldwide to study therapeutics potentially useful in treating MS as well as studying a model of MS. EAE is also useful as a model of inflammation and the advantages of using EAE are that the inflammation is localized to the central nervous system and that there can be precise control of the induction of EAE since the condition can be established by immunization of susceptible animal strains with whole myelin, myelin-derived proteins and peptides, or synthetic peptides. In addition, results can be obtained relatively quickly using the EAE models as animals are monitored for a period not exceeding 4 to 6 weeks, thus allowing for fast screening of compounds which could be useful in treating MS.

EAE can be induced in SJL mice by subcutaneous immunization with a peptide from proteolipid protein (i.e. PLP₁₃₉₋₁₅₁) in complete adjuvant. After 1 and 3 days, the mice are injected intravenously with 10⁹ heat-killed Bordetella pertussis bacteria to increase the permeability of the blood-brain barrier. EAE develops as follows:
1. Activation of T cells by macrophages and dendritic cells that present PLP₁₃₉₋₁₅₁
2. Elevated expression of interleukin-12 in macrophages and dendritic cells.
3. Differentiation of T cells into effector cells that secrete pro-inflammatory cells and express unique chemokine receptors
4. Increased permeability of the blood-brain-barrier
5. Migration of effector cells and monocytes into brain parenchyma against a gradient of chemokines
6. Local (re-)activation of inflammatory cells
7. Release of mediators of inflammation and destruction of oligodendrocytes and myelin.

A typical EAE disease pattern in SJL mice is shown in FIG. 6. Clinical symptoms develop starting approximately on day 11 after immunization. These symptoms include decrease in body weight and the development of paresis and paralysis. After recovery from the first relapse, several relapses and remissions may occur in about 65% of the animals. Eventually, the paralytic symptoms are chronic in nature.

Experimental Procedures

Liposomes as described in Example 1 were prepared and provided as a sterile stock solution containing 1×10¹⁴ liposomes/ml and stored at 4° C. until use. The stock solution was diluted to a concentration of 1.8×10⁷ liposomes/ml in saline. Pathogen free female SJL mice (Age: 9-12, weight: 16-20 grams; Harlan) were acclimatized for 13 days prior to the start of the study, housed under clean conventional conditions, and were randomized over the treatment groups. The mice were divided into three groups of 12 mice each:
a) Saline (day 0 to day 5);
b) Treatment group 1: 6×10⁵ liposomes (day 0 to day 5); and
c) Treatment group 2: 6×10⁵ liposomes (day 20 to day 25).

All mice were injected intramuscularly with 50 μl of either the saline or the liposome solution in alternating hind legs.

To induce EAE, all mice received subcutaneous injections of 75 μg PLP₁₃₉₋₁₅₁ (Isogen Bioscience B.V.) in a 200 μl emulsion (1:1) of phosphate-buffered saline and complete H37 Ra adjuvant (Lot. 2116643, Difco Laboratories, USA), and was distributed over four sites in the flanks of the mice. The mice also received intravenous injections of 10⁹ Bordetella pertussis bacteria (National Institute for Public Health, Bilthoven, The Netherlands) on days 1 and 3.

All mice were monitored for a total of 42 days. Daily measurements of body weight and disability score were taken to evaluate the clinical signs of EAE. Animals were considered to be affected by EAE when a cumulative score of at least 3 was reached within a period of three consecutive days. The maximum weight loss, maximum EAE and cumulative EAE score was calculated for each mouse. In addition to the total monitoring period, the maximum and cumulative EAE scores were separately determined for the first and second phases of EAE (defined as days 0-20 and days 21-42 respectively) for the mice. In addition, the mean EAE score was determined for the early second phase of EAE (days 21-31) and the late second phase of EAE (days 32-42), which late second phase approximates RRMS phase of MS. A Kruskal-Wallis test was performed on the data to determine significance, and where significance was found, the Dunn's Multiple Comparison Test was used to determine the significance between the different groups.

The following scoring system was used to monitor the degree of disability in the EAE model (Kono et al., J Exp Med 168, 213-227, 1988):

Disability scoring system to determine the severity of EAE

| | |
|---|---|
| 0 | no disease |
| 0.5 | tail paresis or partial paralysis |
| 1 | complete tail paralysis |
| 2 | paraparesis: limb weakness and tail paralysis |
| 2.5 | partial limb paralysis |
| 3 | complete hind- or front limb paralysis |
| 3.5 | paraplegia |
| 4 | quadriplegia, moribund |
| 5 | death due to EAE |

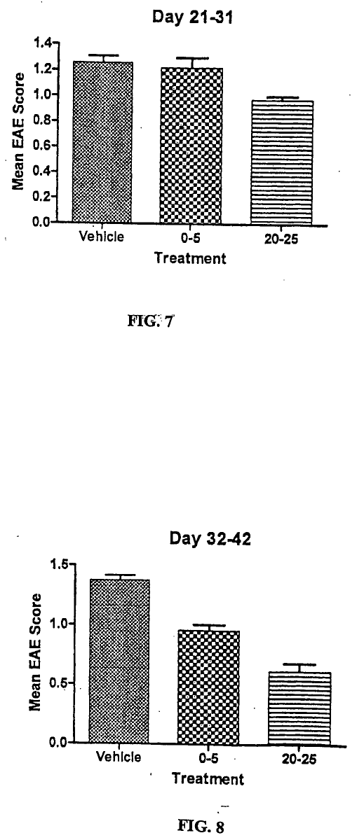

All vehicle treated mice developed EAE, while 10 of 12 mice from Treatment group 1, and all Treatment group 2 mice developed EAE. With respect to the early second phase of EAE, there was a significant decrease in the mean EAE score for mice in Treatment group 2 in comparison to the vehicle control (p≦0.001) while there was no significant difference between Treatment group 1 and the vehicle control (FIG. 7), which indicates that treatment with the PG liposomes at days 20-25 has an effect in decreasing the severity of symptoms of EAE. With respect to the late second phase of EAE, there was a significant reduction in the mean EAE score of Treatment groups 1 (<0.05) and 2 (p<0.001) in comparison to vehicle control (FIG. 8), indicating that treatment with liposomes lessens the severity of the clinical symptoms in this model during a relapse, with treatment at days 20-25 having a relatively greater effect in lessening the severity of the clinical symptoms in this model during a relapse than treatment at days 0-5.

The reduction in the mean EAE score in the treatment groups indicates that treatment with the liposomes affects the clinical symptoms of EAE, thus indicating potential use of these compositions as a prophylaxis or treatment for MS.

1-16. (canceled)

17. A method for reducing pathological damage and/or symptoms associated with multiple sclerosis in a human patient, comprising administering to the patient an effective amount of phosphatidyl glycerol (PG)-carrying bodies.

18. The method according to claim 17, wherein the PG-carrying bodies are liposomes constituted to the extent of at least 10% by weight of phosphatidylglycerol.

19. The method according to claim 18, wherein the liposomes are constituted to the extent of at least 50% by weight of phosphatidylglycerol

20. The method according to claim 18, wherein the liposomes are constituted to the extent of 60%-100% by weight of phosphatidylglycerol.

21. The method according to claim 18 wherein the liposomes are constituted to the extent of 70%-90% by weight of phosphatidylglycerol.

22. The method according to claim 18 wherein the liposomes are constituted to the extent of 75% by weight of phosphatidylglycerol.

23. The method according to claim 17, wherein the PG-carrying bodies have a diameter of from about 20 nanometers to about 500 micrometers

24. The method according to claim 23 wherein the PG-carrying bodies have a diameter from about 20 nanometers to about 1000 nanometers.

25. The method according to claim 23 wherein the PG-carrying bodies have a diameter of from about 20 nanometers to about 500 nanometers.

26. The method according to claim 23 wherein the PG-carrying bodies have a diameter of from about 20 nanometers to about 200 nanometers.

27. The method according to claim 17, wherein the PG-carrying bodies are administered in a unit dosage amount of from about 500 to about 2.5×10¹² bodies.

28. The method according to claim 27 wherein the PG-carrying bodies are in a unit dosage amount of from about 5000 to about 500,000,000 bodies.

29. The method according to claim 27 wherein the PG-carrying bodies are in a unit dosage amount of from about 10,000 to about 10,000,000 bodies.

30. The method according to claim 27 wherein the PG-carrying bodies are in a unit dosage amount of from about 200,000 to about 2,000,000 bodies.

31. The method according to claim 17, wherein the PG-carrying bodies are administered intramuscularly.

32. The method according to claim 17, wherein the patient has secondary progressive multiple sclerosis.

33. The method according to claim 17, for the prophylaxis or treatment of multiple sclerosis.

34. A method of combating multiple sclerosis in a human patient, comprising administering to the patient, a therapeutically effective amount of phosphatidylglycerol (PG)-carrying bodies.

35. The method according to claim 34 wherein the PG-carrying bodies are liposomes constituted to the extent of at least 10% by weight of phosphatidylglycerol.

36. The method according to claim 35 wherein the liposomes are constituted to the extent of at least 50% by weight of phosphatidylglycerol.

37. The method according to claim 35 wherein the liposomes are constituted to the extent of 60%-100% by weight of phosphatidylglycerol.

38. The method according to claim 35 wherein the liposomes are constituted to the extent of 70%-90% by weight of phosphatidylglycerol.

39. The method according to claim 35 wherein the liposomes are constituted to the extent of 75% by weight of phosphatidylglycerol.

40. The method according to claim 34, wherein the PG-carrying bodies have a diameter of from about 20 nanometers to about 500 micrometers.

41. The method according to claim 40 wherein the PG-carrying bodies have a diameter from about 20 nanometers to about 1000 nanometers.

42. The method according to claim 40 wherein the PG-carrying bodies have a diameter of from about 20 nanometers to about 500 nanometers.

43. The method according to claim 40 wherein the PG-carrying bodies have a diameter of from about 20 nanometers to about 200 nanometers.

44. The method according to claim 34, wherein the PG-carrying bodies are administered in a unit dosage amount of from about 500 to about 2.5×10¹² bodies.

45. The method according to claim 44 wherein the PG-carrying bodies are in a unit dosage amount of from about 5000 to about 500,000,000 bodies.

46. The method according to claim 44 wherein the PG-carrying bodies are in a unit dosage amount of from about 10,000 to about 10,000,000 bodies.

47. The method according to claim 44 wherein the PG-carrying bodies are in a unit dosage amount of from about 200,000 to about 2,000,000 bodies.

48. The method according to claim 34, wherein the PG-carrying bodies are administered intramuscularly.

49. The method according to claim 34, wherein the patient has secondary progressive multiple sclerosis.

50. The method according to claim 34 for reducing symptoms associated with multiple sclerosis in a human patient
